(19) United States
(12) Patent Application Publication
(10) Pub. No.: US 2014/0212328 A1
(43) Pub. Date: Jul. 31, 2014

(54) AIR PURIFICATION

(71) Applicants: William Brenden Carlson, Seattle, WA (US); Gregory David Phelan, Cortland, NY (US)

(72) Inventors: William Brenden Carlson, Seattle, WA (US); Gregory David Phelan, Cortland, NY (US)

(73) Assignee: EMPIRE TECHNOLOGY DEVELOPMENT LLC, Wilmington, DE (US)

(21) Appl. No.: 13/988,313

(22) PCT Filed: Jul. 24, 2012
(86) PCT No.: PCT/US12/48016

§ 371 Date: May 17, 2013

(51) Int. Cl. A61L 9/00; D01D 5/04; D01D 5/08; D01D 5/06

(52) U.S. Cl. CPC A61L 9/00; D01D 5/06; D01D 5/04; D01D 5/08
USPC 422/4; 422/120; 546/4; 548/103; 549/265; 549/289; 540/145; 526/241; 525/326.6; 264/178 F; 264/205; 264/211.1

(57) ABSTRACT

Embodiments herein generally relate to the use, devices, and compounds for generating singlet oxygen. In some embodiments, the singlet oxygen can be used for fluid purification and/or sterilization.

### TECHNICAL FIELD

Some embodiments provided herein generally relate to purification devices and methods.

### BACKGROUND

A variety of filters and filtering techniques exist for the purification of various fluids, such as air and liquids. In some situations, such filters are relatively inert and filter the fluid by physically removing particulates from the fluid.

### SUMMARY

In some embodiments, an air filter is provided. The air filter can include a polymer fiber that includes a polymer that includes a monomer unit of a material capable of excited state energy transfer and a polymerized moiety covalently attached to the material capable of excited state energy transfer.

In some embodiments, a method for decontaminating a volume of material is provided. The method can include providing a polymer that includes a monomer unit that includes a material capable of excited state energy transfer and a polymerized moiety covalently attached to the material capable of excited state energy transfer. The method can further include generating at least one singlet oxygen from the polymer and oxygen. The method can further include contacting a volume of material with the singlet oxygen, thereby decontaminating the volume of material.

In some embodiments, a method of making a polymer fiber is provided. The method can include providing a polymer including a material capable of excited state energy transfer covalently attached to a polymerized moiety and forming one or more fiber from the polymer.

In some embodiments, a polymerizable monomer is provided. The polymerizable monomer can include the structure as represented in Formula I, Formula II, Formula III, Formula IV, Formula V, Formula VI, Formula VII, or Formula VIII:

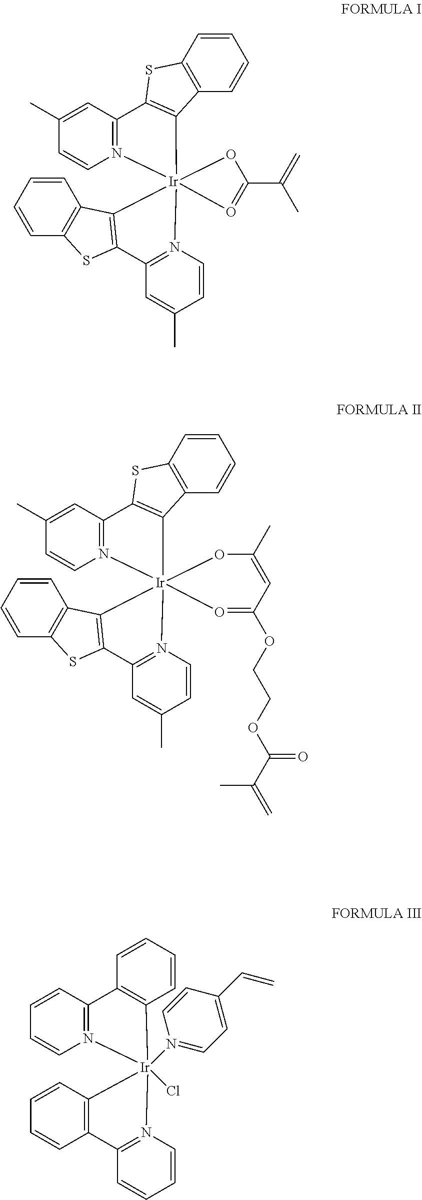

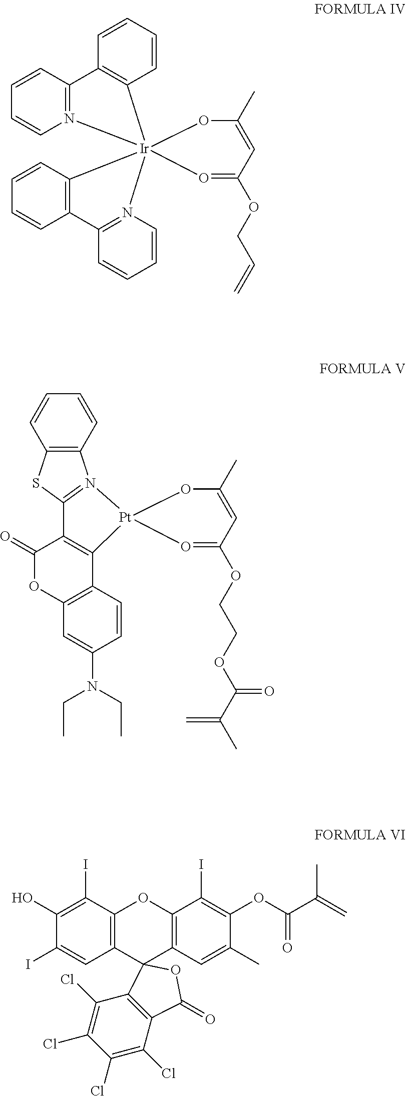

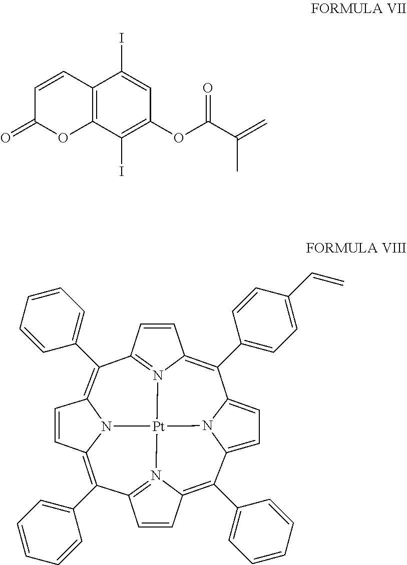

In some embodiments, a polymer including one or more monomer units is provided. In some embodiments, the monomer units are represented by Formula IX:

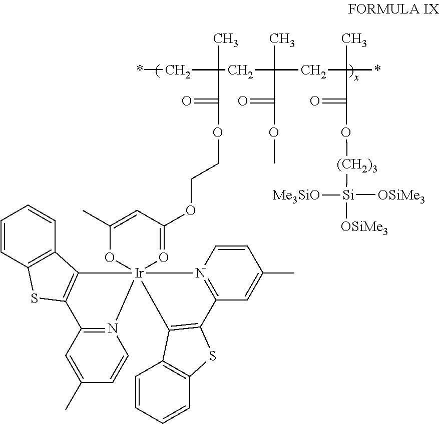

where x can be any number of repeating units, for example 1 to 1 million. In some embodiments, the polymer can include random copolymers of the monomers provided herein. In some embodiments, the polymer can include block polymers of the monomers provided herein. In some embodiments, the polymer can include a random block A and a random block B where random block A can include poly(methyl methacrylate) with iridium containing monomers mixed in and the random block B can include the acrylic silane with iridium containing monomers mixed in. In some embodiments, the polymer includes any one or more of the monomers noted herein. In some embodiments, the polymer includes a singlet oxygen generating (“SOG”) moiety and/or monomer unit.

In some embodiments, a polymer fiber is provided. The fiber can include a polymer including a monomer unit of a material capable of transferring energy from a triplet state of the material to a triplet state of oxygen and a polymerized moiety covalently attached to the material. In some embodiments, the fiber can be configured for use in a filter or contained within or as part of a filter.

In some embodiments, a polymer fiber is provided. The polymer fiber can include a polymer including a monomer unit of a singlet oxygen generating material (“SOG”), and a polymerized moiety covalently attached to the singlet oxygen generating material.

In some embodiments, an air filter material is provided. The air filter material can include a material including a metal chelate moiety of Formula XXXIII

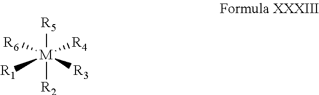

M is at least one of iridium, copper, nickel, tin, lead, europium, gadolinium, samarium, terbium, neodymium, thorium, uranium, rhenium, osmium, ruthenium, rhodium, platinum, silver, palladium, gold, cadmium, or mercury. R₁, R₂, R₃, R₄, R₅, and R₆ are each independently selected from the group of B, C, N, O, F, Si, P, S, Cl, Ge, As, Se, Br, Sn, Sb, Te, or I. R₁, R₂, R₃, R₄, R₅, and R₆ can, optionally, be covalently bonded to one or more of R₁, R₂, R₃, R₄, R₅, and R₆. At least one of R₁, R₂, R₃, R₄, R₅, and R₆ is attached to a polymerizable moiety or part of an entity attached to a polymerizable moiety.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a drawing of some embodiments of an air filter.

FIG. 2 is a flow chart of some embodiments of a method of purifying a volume of material, such as a fluid.

FIG. 3 shows an example of a reaction scheme for making some embodiments of a singlet oxygen generating moiety that can be polymerized. The synthesis scheme is for an acrylic functional iridium based singlet oxygen generator, Ir(MeBTP)₂MMAc.

FIG. 4 shows an example of a reaction scheme for making some embodiments of a singlet oxygen generating moiety that can be polymerized. The synthesis scheme is of an acrylic-acetylacetate functional iridium based singlet oxygen generator, Ir(MeBTP)₂AAc.

FIG. 5 is a graph depicting absorbance and emission aspects of an iridium type complex, such as shown in FIG. 3 and FIG. 4.

FIG. 6 shows an example of a reaction scheme for making some embodiments of a singlet oxygen generating moiety that can be polymerized. The synthesis scheme is of a styrenic functional iridium based singlet oxygen generating moiety, Ir(ppy)₂VpyCl.

FIG. 7 shows an example of a reaction scheme for making some embodiments of a singlet oxygen generating moiety that can be polymerized. The synthesis scheme is of a vinyl functional iridium based singlet oxygen generating moiety, Ir(ppy)₂(vacac).

FIG. 8 shows an example of a reaction scheme for making some embodiments of a singlet oxygen generating moiety that can be polymerized. The synthesis scheme is of an acrylic functional platinum based singlet oxygen generating moiety.

FIG. 9 shows an example of a reaction scheme for making some embodiments of singlet oxygen generating polymers. The singlet oxygen generating moieties polymer includes a methyl methacrylate, a silane methacrylate, and an acrylic functional iridium complex that generates singlet oxygen.

FIG. 10 depicts formulae of some embodiments of singlet oxygen generating moieties.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Provided herein are embodiments that relate to fluid purification. Rather than merely relying on physical separation of contaminants from various fluids, some embodiments that are provided herein allow for the use of singlet oxygen for fluid purification. In some embodiments, this can be achieved by the use of a singlet oxygen generating moiety. In some embodiments, the singlet oxygen generating moiety can be polymerizable and/or part of a polymerized molecule. Thus, in some embodiments, a polymerized and/or polymerizable form of a singlet oxygen generating moiety is provided. For example, this can include a polymer and/or polymerizable molecule that is covalently attached to a moiety that is capable of excited state energy transfer from a triplet state of the moiety to a triplet state of oxygen. Thus, provided herein are embodiments relating to monomers and polymers that include singlet oxygen generating (“SOG”) moieties and uses thereof for the purification of various fluids, such as air and water. Provided herein are embodiments of singlet oxygen generating moieties that are functionalized such that they can be incorporated as, and/or into, a polymer.

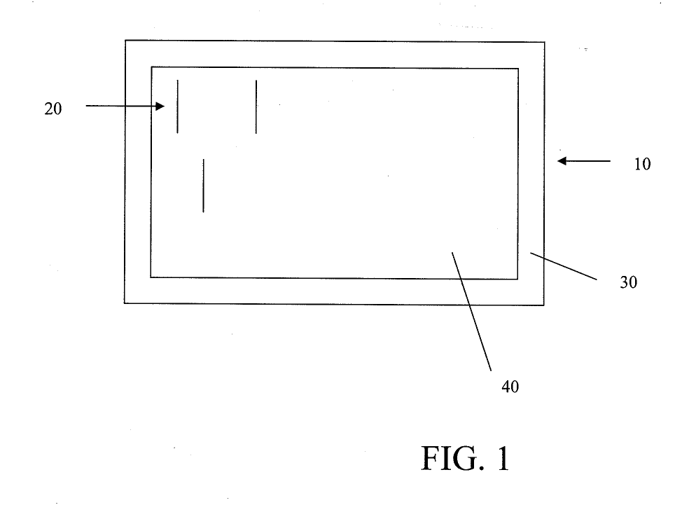

While the singlet oxygen generating moieties can be used in a wide variety of applications, in some embodiments, the singlet oxygen generating moieties can be employed in a fluid filter, such as an air filter. FIG. 1 displays some embodiments of such a filter. The filter 10 can include one or more singlet oxygen generating moieties 20, which can be in polymer form. The polymer can include a monomeric unit of a material capable of excited state energy transfer and a polymerized moiety covalently attached to the material capable of excited state energy transfer. In some embodiments, the filter can include a frame 30 that can support a fluid permeable support 40, through which the fluid to be treated can pass. While, in some embodiments, the fluid permeable support 40 can act to physically filter the fluid, it need not do so in all embodiments, as the presence of singlet oxygen generated from the singlet oxygen moieties can provide sterilizing and/or purifying aspects to the filter.

In some embodiments, the support 40 can be made from plastic, paper, cellulose, carbon, graphite, sol gel, titanate, zirconate, quartz, mineral, plaster, calcite, lime, ceramic, metal, glass, wood, zeolite, cloth, fabric, fluorinated polymer weave material, polymeric weave material, and/or a polymer. In some embodiments, the support 40 can be made entirely, or in part, of a singlet oxygen generating moiety containing polymer. In some embodiments, the singlet oxygen generating moiety containing polymer can cover at least 1% of the surface of the support 40, for example, 10, 20, 30, 40, 50, 60, 70, 80, 90, or 100% of the surface of the support can include the singlet oxygen generating moiety containing polymer. In some embodiments, the support is effectively inert to singlet oxygen. In some embodiments, the support is flexible and/or rigid. In some embodiments, the support includes a porous surface. In some embodiments, the support includes a screen and/or sieve and the polymer can be associated with the weave of the screen itself and/or be placed across the weave of the screen, so as to provide a finer filtering ability. In some embodiments, the support includes functionalized groups so as to allow the covalent attachment of a singlet oxygen generating moiety and/or polymer thereof to the support, such as at least one of an amine, a hydroxyl, a glycidyl, an oxetane, a trifluorovinyl ether, a cyanate, an isocyanate, an alkyne, a silane, an azo, a triazine, and/or an azide. In some embodiments, the support allows for the physical association of the moiety and/or polymer to the support, such that the singlet oxygen generating moiety will remain associated with the filter surface.

In some embodiments, the singlet oxygen generating moiety and/or a polymer form thereof can be used in any type of filter. In some embodiments, the filter and/or filter system is one in which singlet oxygen will be effective in neutralizing contaminants that are expected to be present in the fluid to be filtered.

In some embodiments, the frame 30 can be made from any material. In some embodiments, the frame 30 is a rigid, self-supporting structure. In some embodiments, the frame 30 is flexible. In some embodiments, the frame 30 includes metal, plastic, ceramic, paper, cellulose, etc. In some embodiments, the frame is relatively inert to singlet oxygen. While the filter 10 in FIG. 1 displays a simple frame and support arrangement, other arrangements are also applicable. For example, in some embodiments, additional supporting structures can be employed across the support 40, so as to add additional mechanical strength to the support.

In some embodiments, any material capable of excited state energy transfer can be used as the singlet oxygen generating moiety and/or within a singlet oxygen generating polymer. In some embodiments, the material capable of excited state energy transfer can include a material capable of causing a triplet state to be excited in oxygen. In some embodiments, the material capable of causing a triplet state includes at least one of a metal or an organic molecule. In some embodiments, the metal includes at least one of iridium, copper, nickel, tin, lead, rhenium, osmium, ruthenium, rhodium, platinum, silver, palladium, gold, cadmium, or mercury. In some embodiments, the metal includes iridium. In some embodiments, the organic molecule includes at least one of coumarin, fluorescein, or rhodamine. In some embodiments, the organic molecule can also include iodine, bromine, tellurium, selenium, aluminum, gadolinium, antimony, pyrene, benzopyrene, perylene, terrylene, quaterrylene, pentatrylene, hexatrylene, hepatrylene, octarylene, fluorene, vinyl carbazole, thiazole, phenylene oxide, N,N,N′,N′-tetramethylacridine-3,6-diamine, 2,7-dimethylacridine-3,6-diamine and acrylamides thereof, (meth)acrylates of fluorescein or a combination thereof. These options can be attached to, and/or part of, the organic molecule. In some embodiments, the organic molecule includes a brominated or iodoated fluorescent dye. In some embodiments, the fluorescent dye that can be brominated or iodoated can include Acridine dyes, Cyanine dyes, Fluorone dyes, Oxazin dyes, Phenanthridine dyes, or Rhodamine dyes. In some embodiments, the fluorescent dye that can be brominated or iodoated can include Acridine orange, Acridine yellow, Alexa Fluor, 7-Aminoactinomycin D, 8-Anilinonaphthalene-1-sulfonate, ATTO dyes, Auramine-rhodamine stain, Benzanthrone, Bimane, 9,10-Bis(phenylethynyl)anthracene, 5,12-Bis(phenylethynyl)naphthacene, Blacklight paint, Brainbow, Calcein, Carboxyfluorescein, Carboxyfluorescein diacetate succinimidyl ester, Carboxyfluorescein succinimidyl ester, 1 -Chloro-9,10-bis(phenylethynyl)anthracene, DyLight Fluor, Ethidium bromide, Fluo-4, FluoProbes, Fluorescein, Fluorescein isothiocyanate, Fluoro-Jade stain, Fura-2, Fura-2-acetoxymethyl ester, Green fluorescent protein, Heptamethine dyes, Hoechst stain, Indian yellow, Indo-1, Lucifer yellow, Luciferin, Phycoerythrin, Phycoerythrobilin, Propidium iodide, Pyranine, Rhodamine, Rhodamine 123, Rhodamine 6G, RiboGreen, Rubrene, (E)-Stilbene, Z)-Stilbene, Sulforhodamine 101, Sulforhodamine B, SYBR Green I, Synapto-pHluorin, Tetraphenyl butadiene, Tetrasodium tris(bathophenanthroline disulfonate)ruthenium(II), Texas Red, Titan yellow, TSQ, 2-Chloro-9,10-bis(phenylethynyl)anthracene, 2-Chloro-9,10-diphenylanthracene, Coumarin, DAPI, Dark quencher, DiOC6, MCherry, Merocyanine, Nile blue, Nile red, Optical brightener, Perylene, Phloxine, Phycobilin, or Umbelliferone.

In some embodiments, the air filter material can include a material including a metal chelate moiety of Formula XXXIII.

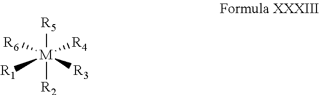

M is at least one of iridium, copper, nickel, tin, lead, europium, gadolinium, samarium, terbium, neodymium, thorium, uranium, rhenium, osmium, ruthenium, rhodium, platinum, silver, palladium, gold, cadmium, or mercury. R₁, R₂, R₃, R₄, R₅, and R₆ are each independently selected from the group of B, C, N, O, F, Si, P, S, Cl, Ge, As, Se, Br, Sn, Sb, Te, or I. R₁, R₂, R₃, R₄, R₅, and R₆ can, optionally, be covalently bonded to one or more of R₁, R₂, R₃, R₄, R₅, and R₆. At least one of R₁, R₂, R₃, R₄, R₅, and R₆ is attached to a polymerizable moiety, is a polymerizable moiety, or is attached to a part of an entity attached to a polymerizable moiety. In some embodiments, one or more of R₁, R₂, R₃, R₄, R₅, and R₆ can be covalently linked to one or more of R₁, R₂, R₃, R₄, R₅, and R₆. Thus, in such embodiments, R₁, R₂, R₃, R₄, R₅, and R₆ can, for example, at least partially surround M in a linear, bent, trigonal planar, square planar, tetrahedral, trigon bipyramidal, octaheadral, pentagonal bipyramidal, square antiprismatic, bisdisphenoid, or hexagonal bipyramidal arrangement. The polymerizable moiety can be any of the polymerizable moieties provided herein, such as an acrylic group, a styrenic group, polyurethane, polyurea, a vinyl group, acryclic, acrylic-acetate, methacrylate, styrene, vinylic moieties, vinyl ketone, vinyl ether, vinyl amine, urethane, urea, polyester, polyether, polycarbonate, and/or epoxies. Furthermore, in some embodiments, the material including the metal chelate moiety of Formula XXXIII is in its polymerized form.

In some embodiments, the singlet oxygen can be produced by energy transfer from the triplet state of the moiety to the triplet state of oxygen. In some embodiments, the singlet oxygen can deactivate in a variety of ways, for example, it can collide with an object and it oxidizes that object; it can transfer energy to that object and regenerates triplet ground state oxygen; or it can split off a quanta of energy and reform ground state oxygen. In some embodiments a singlet oxygen quencher can be employed so as to restrict the presence of the singlet oxygen. In some embodiments, any type of singlet oxygen quencher can be used, for example thioredoxin.

In some embodiments, the singlet oxygen generating metal can be a neutral compound. In some embodiments, the metal can include salts with counter ions. In some embodiments any of the complexes and/or organic molecules can be charged and that charge can be balanced with a counter ion. In some embodiments, the singlet oxygen generating polymer can be conductive. In some embodiments, the polymer can include an alkyne in lieu of a vinyl so as to allow for conductive polymers. In some embodiments, the polymer includes a singlet oxygen generating moiety and one or more alkyne groups so as to produce a conductive polymer.

In some embodiments, any polymerizable moiety can be associated with the singlet oxygen generating moiety to create a singlet oxygen generating polymer. In some embodiments, the polymerized moiety includes at least one polymerized molecule from the group of: an acrylic group, a styrenic group, alkyne, polyurethane, polyurea, or a vinyl group. In some embodiments, the polymer group is selected from at least one of acryclic, acrylic-acetate, methacrylate, styrene, vinylic moieties, vinyl ketone, vinyl ether, vinyl amine, urethane, urea, polyester, polyether, polycarbonate, vinylformamide, vinyl acetate (and the longer chain derivatives such as propate, butyrate, pentate, hexate and so on), tetrafluoroethylene, trifluoromethyltrifluoroethylene, vinylidene fluoride, vinylidene chloride, vinyl chloride, vinyl ethers, silicones, trimethylsilylpropyne and epoxies.

In some embodiments, the polymer is used to create a larger fiber and the fiber is positioned within the filter, for example within the support 40. In some embodiments, the fiber and/or singlet oxygen generating polymer can be positioned on top of or on a surface of the support. In some embodiments, the polymer is sprayed onto the support. In some embodiments, the support is dipped into a solution containing the singlet oxygen generating polymer and then the support is removed and the solvent evaporated from the support, leaving the support coated with the singlet oxygen generating polymer.

In some embodiments, the filter is at least one of: a commercial air filter, a residential air filter, a catalytic converter, or a water filter.

In some embodiments, the singlet oxygen generating polymer forms at least a part of a fiber. In some embodiments, the fiber forms at least a part of a filter, such as the support 40.

In some embodiments, a method of purifying and/or decontaminating a fluid is provided. In some embodiments, the singlet oxygen generating moiety and/or singlet oxygen generating polymer can be used to generate singlet oxygen and the singlet oxygen is then used for the filtering and/or decontamination of the fluid that is proximal to and/or passes through the filter and/or singlet oxygen generating moiety.

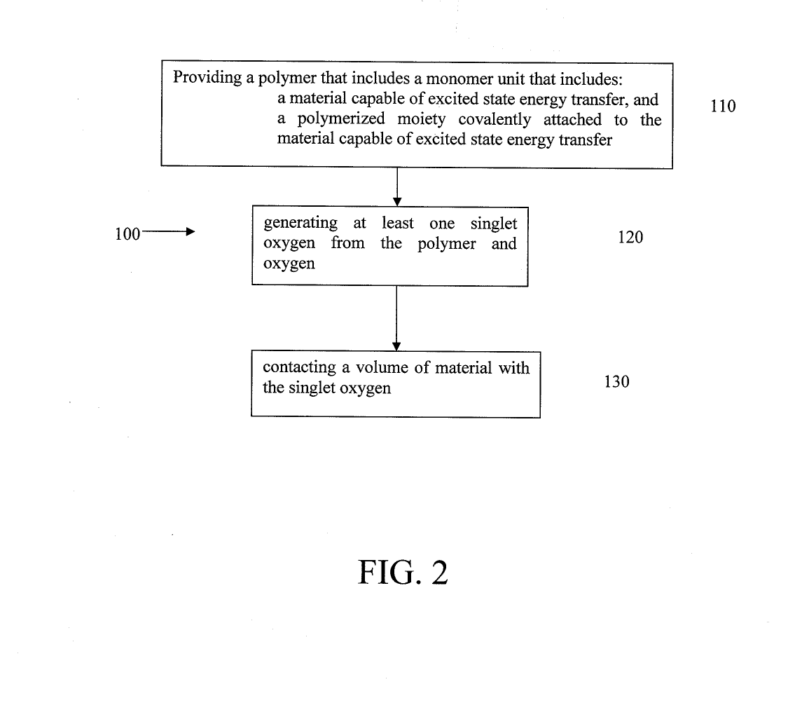

FIG. 2 provides a flow chart of some embodiments of such methods 100. In some embodiments, the method for decontaminating a volume of material can include providing a polymer that includes a monomer unit including a material capable of excited state energy transfer. In some embodiments, the polymer further includes a polymerized moiety that is covalently attached to the material capable of excited state energy transfer (and/or singlet oxygen generation) (block 110). One can further generate at least one singlet oxygen from the polymer and oxygen (block 120) and contact a volume of material (for example a fluid, such as air or water) with the singlet oxygen (block 130). The generated singlet oxygen can then decontaminate the volume of material. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In some embodiments, the singlet oxygen is produced by excited state energy transfer from the singlet oxygen generating moiety to oxygen. The excited state can be sufficiently long-lived to provide adequate time for oxygen to diffuse to the region and collide with the singlet oxygen generating moiety for energy transfer to take place. In some embodiments, while any excited state can be used to produce singlet oxygen, triplet excited states are highly efficient at producing singlet oxygen. In some embodiments, the materials (such as the support) are sufficiently oxygen permeable to allow sufficient quantities of oxygen to reach the singlet oxygen generating moieties such that adequate amounts of singlet oxygen can be produced.

In some embodiments, the material and/or fluid to be filtered can be any type of fluid or flowable material. In some embodiments, the fluid can include air, water, or some combination thereof. In some embodiments, the volume of material and/or fluid to be filtered includes a volume of air and the volume of air is moved through the filter. In some embodiments, the material includes a liquid for consumption.

In some embodiments, decontamination includes the destruction and/or breakdown of at least one chemical agent or biological agent. In some embodiments, the biological agent includes at least one of a bacterium, a parasite, a prion, or a virus. In some embodiments, the agent to be broken down includes one or more of pollen, spores, dichloroethyl sulfide, soman, tabun, sarin, and/or ethyl ({2-[bis(propan-2-yl)amino]ethyl}sulfanyl)(methyl)phosphinate.

In some embodiments, generating singlet oxygen includes exposing the singlet oxygen generating polymer to some form of radiation, including, for example visible light, ultraviolet light, or infrared light. In some embodiments, the visible light includes at least one wavelength of blue or green light. In some embodiments, the filter can include a light source. In some embodiments, the device in which the filter is to be placed can include a light source. In some embodiments, energy to produce the singlet oxygen can be provided via electricity, which can be applied to the singlet oxygen generating moiety and/or polymer. In embodiments in which the polymer itself is conductive, then an electrical potential can be applied directly via the polymer.

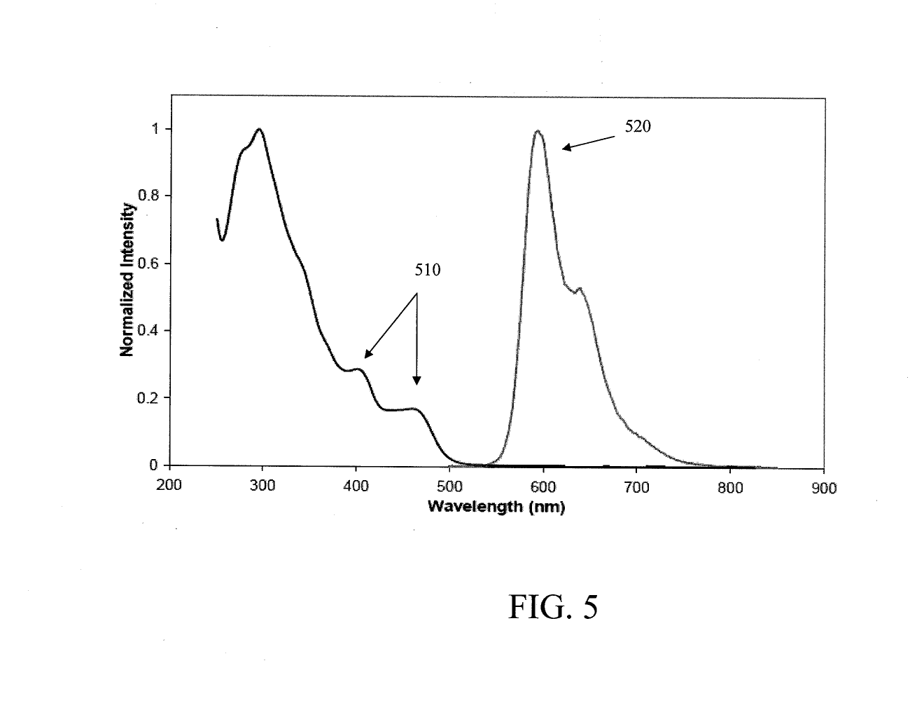

In some embodiments, the method can further include monitoring an amount of singlet oxygen emitted from the filter. In some embodiments, one or more emission bands can be used to monitor the air filter and indicate when the filter is effective at decontaminating the air or when the filter needs to be replaced (see, for example, Example 2 and FIG. 5). In some embodiments, this can be monitored by a photomultiplier and/or photo diode, which can optionally be built into the filter and/or device. In some embodiments, the filter and/or filtering device includes a singlet oxygen detector. In some embodiments, when the singlet oxygen level being produced is not as high as desired for a particular application, additional energy (such as light or electricity) can be applied to the singlet oxygen moieties and/or polymers. In some embodiments, when the singlet oxygen level being produced is not as high as desired for a particular application the filter can be replaced with a new filter. In some embodiments, when the singlet oxygen level being produced is not as high as desired for a particular application, additional singlet oxygen moieties and/or polymers thereof can be applied to the surface 40.

In some embodiments, the method includes enriching an amount of oxygen in the volume of material such that additional singlet oxygen can be produced.

In some embodiments, the decontamination process is part of a commercial air filtration, a resident air filtration, a catalytic conversion, a water filtering, or a biological contamination process, so as to remove various contaminants. In some embodiments, the process is performed in or for a vehicle, such as a car. In some embodiments, the process is performed in an apartment or business. In some embodiments, the process is performed so as to provide purified air to a clean room. In some embodiments, the process is performed so as to provide purified water from and/or as part of a water purification process in a waste water treatment plant.

In some embodiments, the method includes applying an electrical potential to the volume of material to be filtered. The material to be filtered can include air or water. In some embodiments, the electrical potential is applied by a conductive polymer in a filter of which the polymer is part or is combined with in a larger fiber. In some embodiments, the electrical potential is applied via a frame of the filter (for example, electrodes can be part of the frame). In some embodiments, the electrical potential can be applied via the support, for example, when the support is one or more metal screens.

In some embodiments, a single filter can be used to purify the fluid. In some embodiments, multiple filters can be used. In some embodiments, the filters can be in series and be the same, and thereby provide a greater degree of purification. In some embodiments, the filters can be different, and allow for the removal of different types of contaminants. For example, a first, size-based filter can be used to remove larger particulates, and then the fluid can be filtered though the singlet oxygen generating filter afterwards (and/or simultaneously).

In some embodiments, the support of the filter is configured so as to minimize or reduce any slow down in fluid-flow due to the filter. Thus, in some embodiments, the filter can be used for high flow rate purification processes. In some embodiments, the flow rate of the fluid is intentionally kept low, so as to allow more time for the singlet oxygen to interact with any contaminants in the fluid.

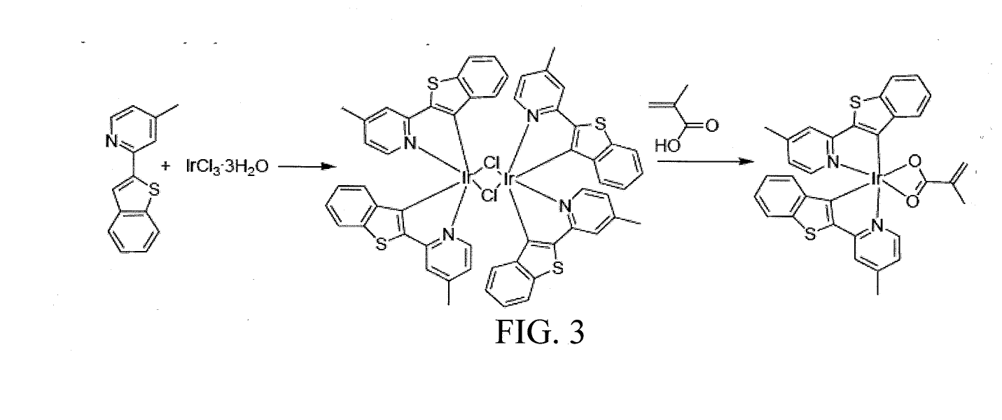

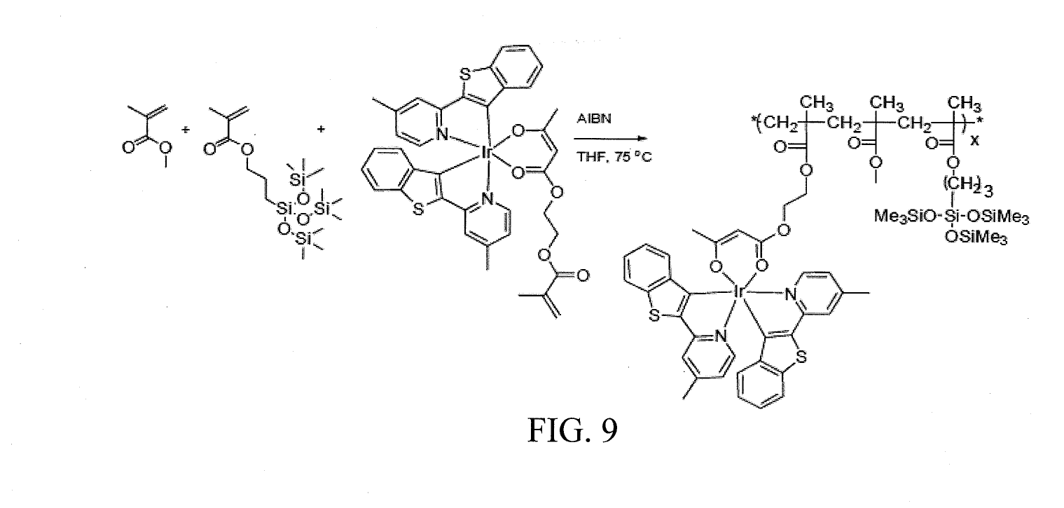

Embodiments provided herein are not limited in their method of manufacture. There are a variety of ways for creating both the singlet oxygen generating moieties, monomers thereof, and polymers thereof. Examples of how to make various monomers of singlet oxygen generating moieties are provided in FIGS. 3, 4, and 6-8 and the Examples below. Examples of how to polymerize the monomers are provided in the examples and FIG. 9. However, in some embodiments, any method of synthesis can be used to create the singlet oxygen generating monomer and/or polymer. In some embodiments, the method can include, for example with iridium, starting with the tris acetylacetone (acac) of Ir, then take the tris acac and react with the organic ligand. The organic ligand then substitutes for the acac to form Ir(organic)₂acac.

In some embodiments, a method of making a polymer fiber is provided. The method can include providing a polymer that includes a material capable of excited state energy transfer (for example, a singlet oxygen generating moiety) covalently attached to (and/or as part of) a polymerized moiety and forming one or more fiber from the polymer. In some embodiments, forming the one or more fiber can include the use of a wet spinning technique. In some embodiments, the wet spinning technique includes placing the material in a liquid in which the polymer is not solvent, placing a spinneret in the solvent, and precipitating the polymer as it emerges from the solvent to form the fiber. In some embodiments, forming the one or more fiber includes dry spinning. In some embodiments, forming the one or more fiber includes a polymer melt.

Any of a variety of singlet oxygen generating moieties or singlet oxygen generating polymers can be employed in the arrangements and methods provided herein. In some embodiments, a polymerizable monomer including the structure as represented in Formula I, Formula II, Formula III, Formula IV, Formula V, Formula VI, Formula VII, or Formula VIII can be used as a monomer to either create a polymer or the unit form within the polymeric form can be employed.

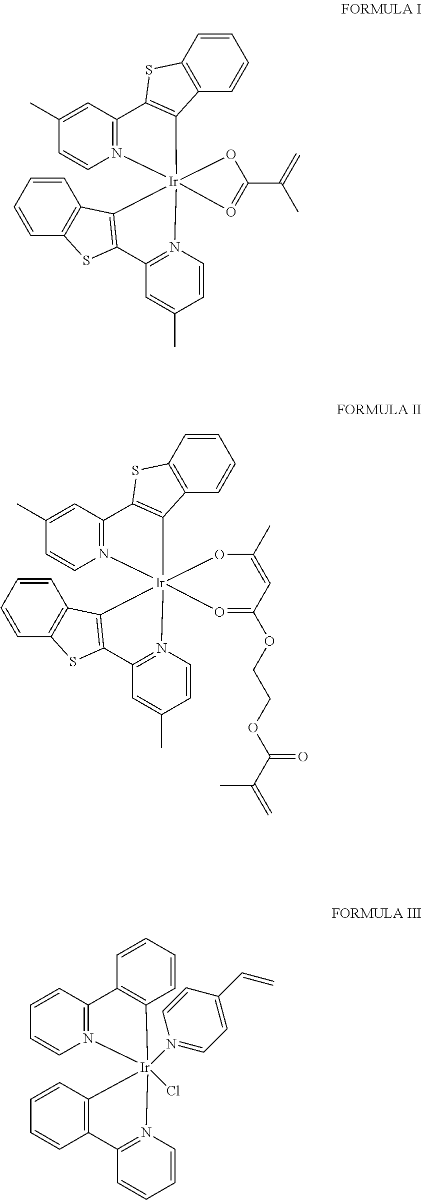

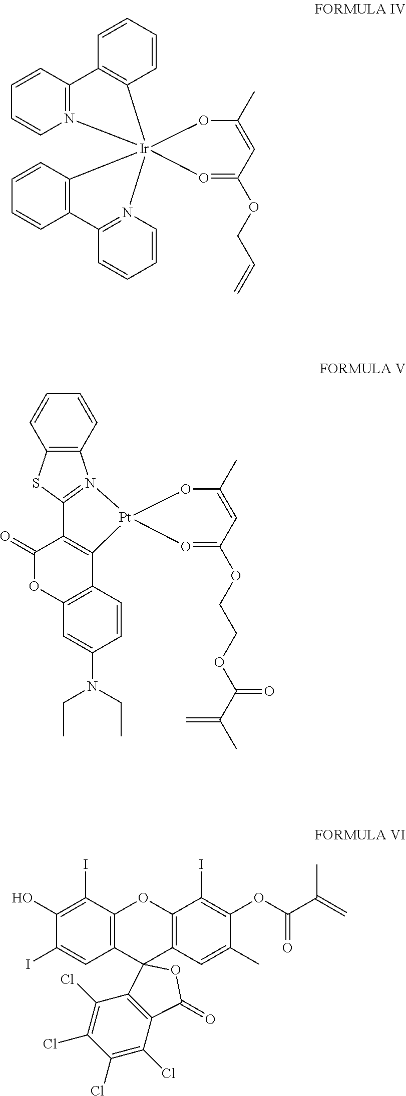

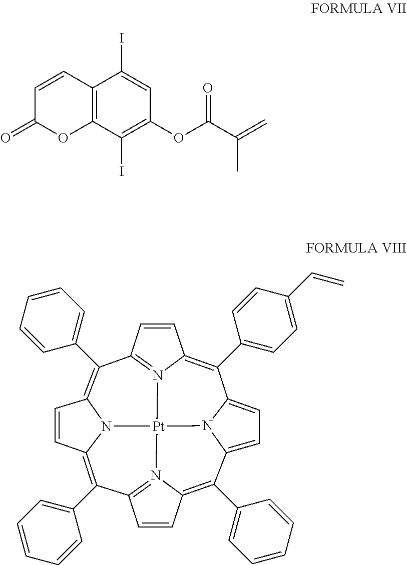

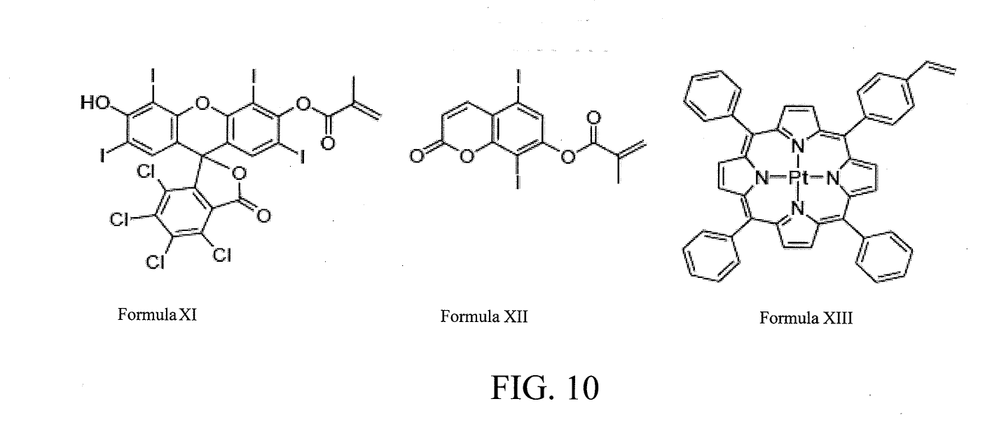

Additional embodiments are also depicted in FIG. 10 as Formula XI, XII, and XIII

In some embodiments, a polymer fiber is provided and can include a polymer including a monomer unit of a material capable of transferring energy from a triplet state of the material to a triplet state of oxygen and a polymerized moiety covalently attached to the material. In some embodiments, the polymer includes a material capable of transferring energy from a triplet state of the material to oxygen. In some embodiments, a polymer fiber is provided and can include a polymer that includes a monomeric unit of a singlet oxygen generating material and a polymerized moiety covalently attached to the singlet oxygen generating material. In some embodiments, the polymer includes singlet oxygen generating moieties that are in polymer form. In some embodiments, the polymer includes monomer units of any one or combination of Formula I, Formula II, Formula III, Formula IV, Formula V, Formula VI, Formula VII, Formula VIII, Formula XI, Formula XII, and/or Formula XIII. In some embodiments, the polymer includes monomer units of any one or combination of the formulae listed below:

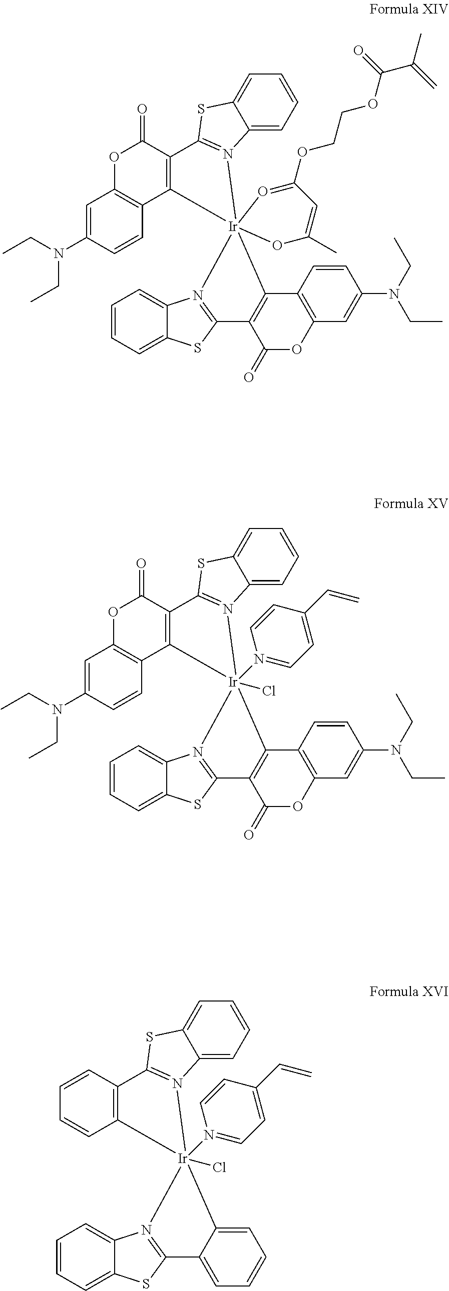

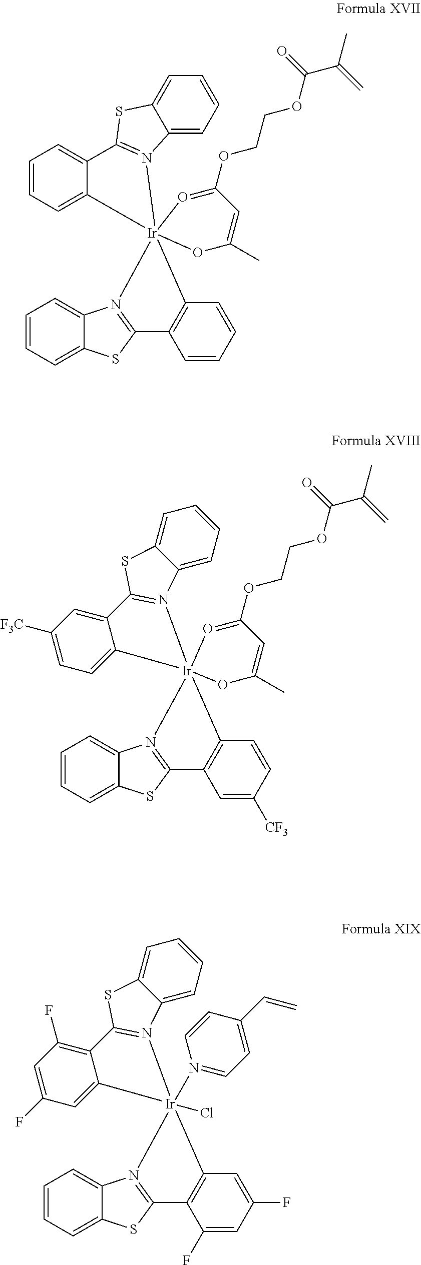

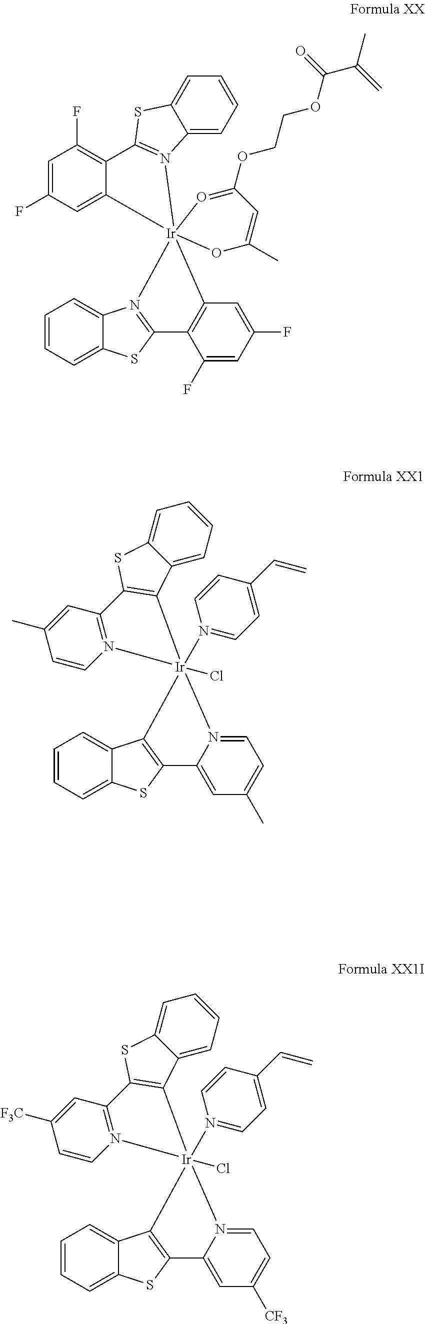

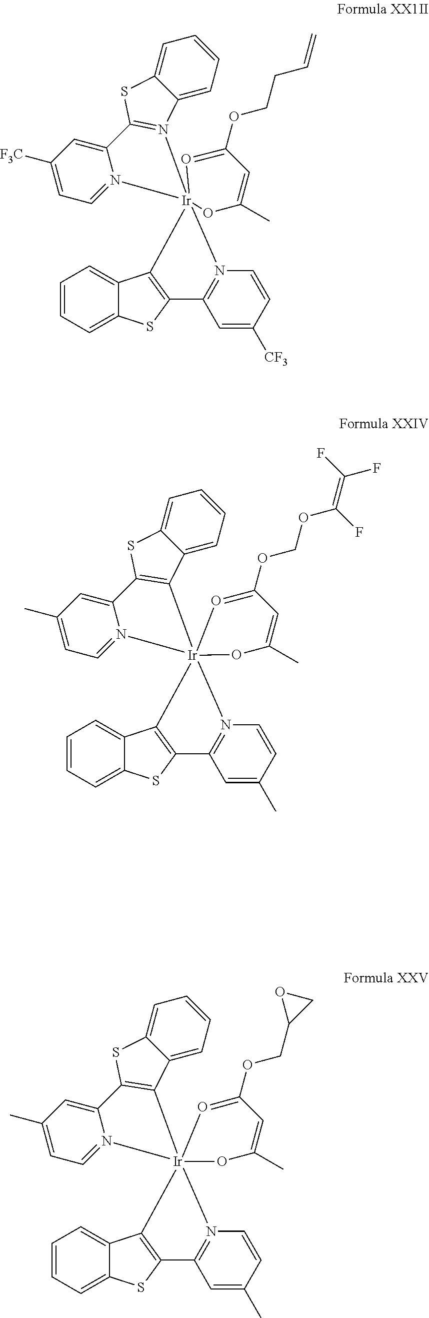

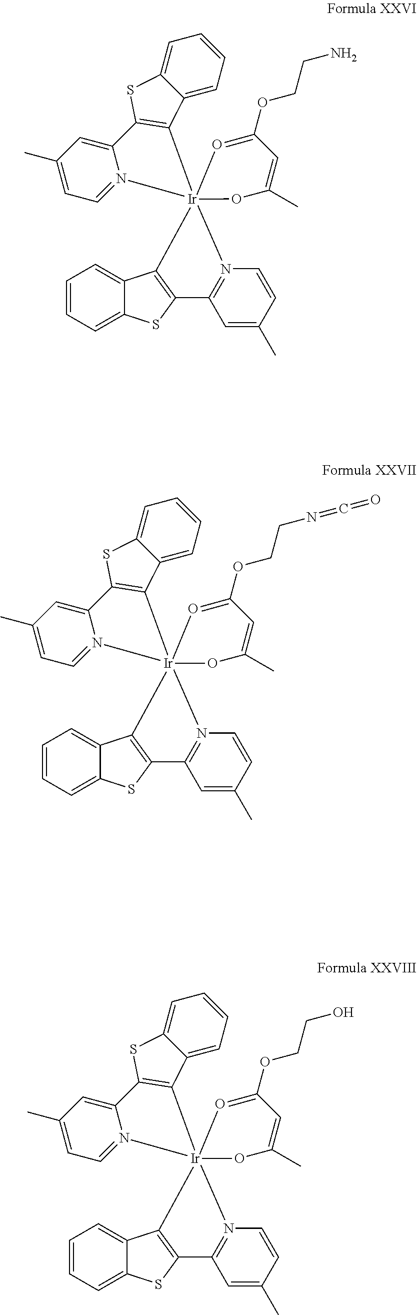

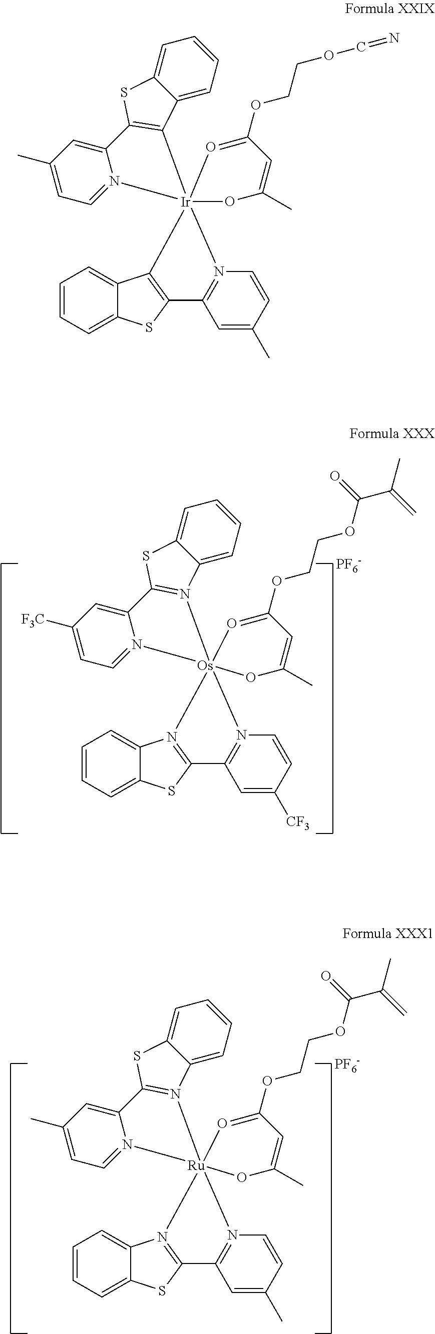

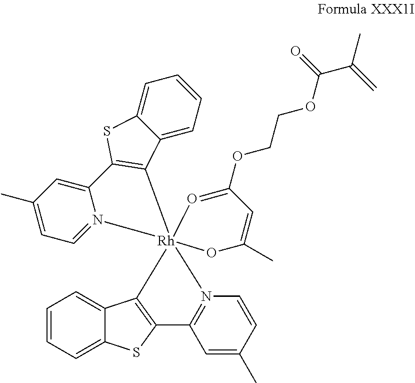

In some embodiments, a singlet oxygen generating polymer is provided. The singlet oxygen generating polymer can include one or more monomer units. In some embodiments, the monomeric units within the polymer are represented by Formula IX:

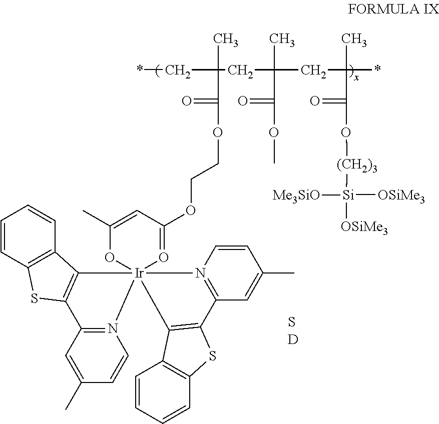

There are a variety of options for incorporating the singlet oxygen generating moieties into a filter and/or fiber. In some embodiments, the singlet oxygen generating moieties and/or singlet oxygen generating polymers are configured in fiber form and can be woven into the filter as another fiber within the filter. In some embodiments, the singlet oxygen generating moieties and/or singlet oxygen generating polymers are embedded and/or coated on a surface of the filter. In some embodiments, the singlet oxygen generating moieties and/or singlet oxygen generating polymers are restrained by a fluid permeable membrane or surface so as to allow the fluid to pass proximally to the singlet oxygen generating moieties and/or singlet oxygen generating polymers. Of course, as the actual purifying aspect can be achieved via the generated singlet oxygen, the fluid need not come into contact with the singlet oxygen generating moieties and/or singlet oxygen generating polymers directly, as long as generated singlet oxygen can come into contact with the fluid.

In some embodiments, filter embodiments provided herein can be made by synthesizing polymerizable singlet oxygen generating moieties into fibers that are then woven into air filter media or some other form for non-woven filter media. Examples for synthesizing various singlet oxygen generating moieties and polymers are provided in the Examples below.

In some embodiments, the fibers themselves (as they can include a significant amount of singlet oxygen generating moieties and/or singlet oxygen generating polymers) produce singlet oxygen to neutralize chemical and biological contaminates that are present in a fluid. In some embodiments, such as when the singlet oxygen generating moieties are in polymer form, phase separation can be adverted and efficient singlet oxygen generation can be maintained. Singlet oxygen generating polymers can allow for greater flexibility in the design of air filtration media as the singlet oxygen generating moieties can be polymerized with a wide variety of other compounds to tune the air filtration system to a variety of applications. While a number of aspects can be considered for this, it is noted that hard glassy polymers tend to add toughness and rigidity to the polymers but tend to reduce oxygen permeability (examples are methyl methacrylate or styrene); fluorinated polymers tend to add chemical resilience and increase oxygen permeability but can increase cost; silicones/silanes tend to greatly increase oxygen permeability, but also allows the polymer to absorb more chemicals; and rubbery materials tend to increase oxygen permeability but tend not to be as oxidatively stable.

In some embodiments, a singlet oxygen generating moiety can be present in a commercial air filtration system or component, such as in filters for air conditioners, filters for heat exchangers, and/or filters for stand alone HEPA devices. In some embodiments, a singlet oxygen generating moiety (including polymers thereof) can be present in a residential air filtration system or component, such as filters for air conditioners, filters for heat exchangers, and/or filters for stand alone HEPA devices.

In some embodiments, a singlet oxygen generating moiety can be used or present in a device for removal of weapons of mass destruction such as radioactive cesium with conductive filter materials and the use of electric potential. In some embodiments, the singlet oxygen generating moiety (and polymers thereof) can be combined with components for or used with filtration materials that decontaminate general biological and chemical contaminates. In some embodiments, the singlet oxygen generating moiety (and polymers thereof) can be combined with components for or used with filtration materials that decontaminate biological and chemical weapons of mass destruction.

In some embodiments, the singlet oxygen generating moiety (and polymers thereof) can be combined with components for, or used with, water filtration. In some embodiments, this can be an individual backpack purification system (for example, for both military and/or civilian use). In some embodiments, the singlet oxygen generating moiety (and polymers thereof) can be combined with components for or used with non-reactive filters. In some embodiments, the singlet oxygen generating moiety (and polymers thereof) can be combined with components for or used with reactive filters. In some embodiments, the singlet oxygen generating moiety (and polymers thereof) can be combined with components for or used with filters to capture radioactive contaminates.

In some embodiments, the singlet oxygen generating moiety (and polymers thereof) can be combined with components for or used with large-scale water purification, such as protection of public water systems from “dirty bomb” and chemical terrorist attacks, bottled water production, and/or de-ionization of water.

In some embodiments, the singlet oxygen generating moiety (and polymers thereof) can be combined with components for or used with extreme surface area catalysts, such as catalytic converters in cars and/or chemical manufacturing.

In some embodiments, one or more of the embodiments provided herein can have one or more of the following aspects: cost effectiveness, uses existing filter system infrastructure, decontaminates biological based agents, decontaminates chemical agents, excellent efficacy at decontamination, decontamination systems can be regenerated, tells when the filter is effective at decontaminating the air and when the filter needs to be replaced, and/or activated with visible light.

In some embodiments, the singlet oxygen generating moiety (and polymers thereof) can be any of those provided herein. In some embodiments, the singlet oxygen generating moiety is polymerizable. In some embodiments, the singlet oxygen generating moiety (and polymers thereof) includes an iridium complex as shown below:

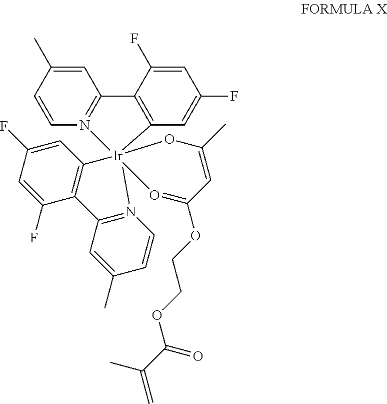

In some embodiments, the singlet oxygen generating moiety (and polymers thereof) containing air filter decontaminates air borne biological and chemical contaminates with high effectiveness and renders them harmless.

In some embodiments, the singlet oxygen generating moiety (and polymers thereof) can be stimulated into greater singlet oxygen production by the addition of energy. In some embodiments, singlet oxygen is continuously produced in the presence of harmless visible light. In some embodiments, there need be no saturation of the singlet oxygen producing species (unlike activated carbon). In some embodiments, the polymerizable singlet oxygen generating moiety reduces phase separation and at least partially maintains efficient singlet oxygen production to assist in providing effective decontamination of biological and chemical agents.

In some embodiments, the singlet oxygen generating moiety (and polymers thereof) can be combined with other filter devices, such as a HPEA filter. In some embodiments, the singlet oxygen generating moiety (and polymers thereof) can be used in a filter to aid in removing and/or neutralizing contaminates such as viruses, mold, mildew, chemicals, and dander dust. In some embodiments, as the singlet oxygen species is highly reactive, it can reduce the presence of air borne agents that might otherwise build on and within a filter because the filtration media does not react with the contaminates to neutralize them.

In some embodiments, the singlet oxygen generated by the singlet oxygen generating moiety (and polymers thereof) can be used to inactivate human viruses and bacterial at contaminated sites and in the processing of fluids against HIV.

In some embodiments, the singlet oxygen generating moiety (and polymers thereof) provided herein allow for the controlled placement of the singlet oxygen generating moieties in the filter material for superior efficacy at neutralization of chemical and biological contaminates in the air of homes and offices.

In some embodiments, the singlet oxygen generating moiety (and polymers thereof) provided herein are effective against both chemical and biological contaminates simultaneously.

In some embodiments, the singlet oxygen generating moiety (and polymers thereof) provided herein reduce the risk of contaminate build up in and on filters by incorporating a singlet oxygen decontamination and a neutralization feature to the filtration materials.

In some embodiments, the singlet oxygen generating moiety (and polymers thereof) provided herein can be monitored by emission output and as such indicate to a user with scientific accuracy when the filter is effective at decontaminating the air and when the filter can be to be replaced.

In some embodiments, the singlet oxygen generating moiety (and polymers thereof) provided herein can be highly effective in either woven or non-woven filtration media.

### EXAMPLES

### Example 1

### Acrylic Functional Iridium Singlet Oxygen Generating Monomer

Iridium chloride hydrate is reacted with 2-Benzo[b]thiophen-2-yl-4-methyl-pyridine (MeBTP) in a solution of 2-ethoxyethanol under a atmosphere of nitrogen for 24 hour to form a chloride-bridged dimer (MeBTP)₂Ir-μ-Cl₂-μ-Ir(MeBTP)₂). The bridged dimer is reacted with (meth)acrylic acid in a solution of 2-methoxyethanol under nitrogen atmosphere at 115 degrees Celsius for approximately 12 hours. After cooling down, the filtrated product is washed with water, hexane, and diethyl ether. The crude product is chromatographed through a silica column with dichloromethane mobile phase. The complex monomer thereby produced, Ir(MeBTP)₂MMAc, can then be polymerized or co-polymerized with various monomers. A schematic of the reaction is shown in FIG. 3.

### Example 2

### Acrylic-acetylacetate Functional Iridium Singlet Oxygen Generating Monomer

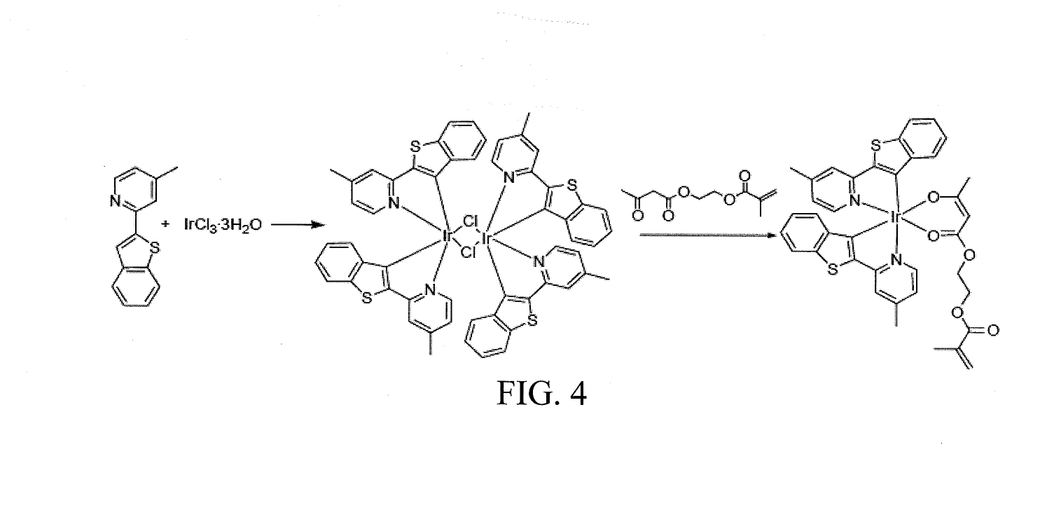

Iridium chloride hydrate is reacted with 2-Benzo[b]thiophen-2-yl-4-methyl-pyridine (MeBTP) in a solution of 2-ethoxyethanol under a atmosphere of nitrogen for 24 hours to form a chloride-bridged dimer (MeBTP)₂Ir-μ-Cl₂-μ-Ir(MeBTP)₂). The bridged dimer is reacted with 3-oxo-butyric acid 2-(2-methyl-acryloyloxy)-ethyl ester (acrylic acetylacetate ligand) in a solution of refluxing 2-methoxyethanol and sodium carbonate under nitrogen atmosphere. After cooling down, the filtrated product is washed with water, hexane, and diethyl ether. The crude product is chromatographed through a silica column with dichloromethane mobile phase. The complex monomer thereby produced, Ir(MeBTP)₂AAc, can then be polymerized or co-polymerized with various monomers. A schematic of the reaction is shown in FIG. 4.

Exemplary absorbance and emission of an iridium type complex as shown in Examples 1 and 2 are displayed in FIG. 5. Absorbance shows prominent visible bands at about 400 and about 450 nm 510 and triplet energy at about 600 nm 520.

In some embodiments, the emission band can be used to monitor the air filter and tell when the filter is effective at decontaminating the air and when the filter needs to be replaced.

### Example 3

### Styrenic Functional Singlet Oxygen Generating Monomer

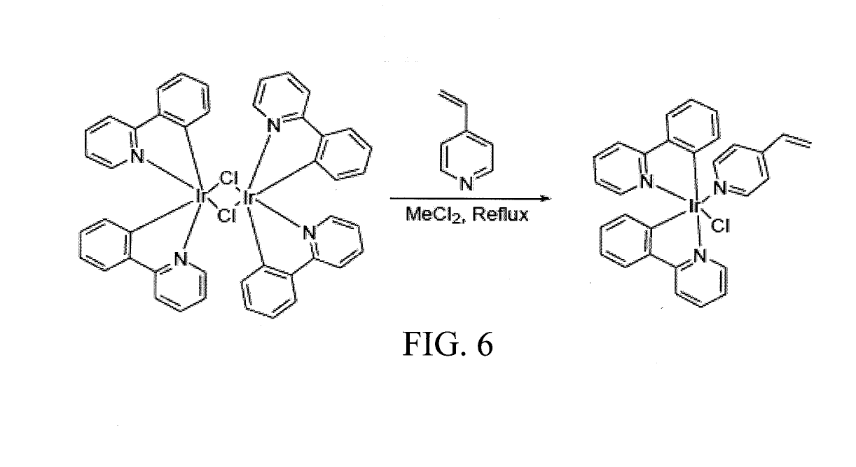

Iridium trichloride trihydrate is reacted as above with 2-phenylpyridine (ppy) to form the bridge dimer complex ((ppy)₂Ir-μ-Cl₂-μ-Ir(ppy)₂). 4-Vinylpyridine (vpy) is added to (ppy)₂Ir-μ-Cl₂-μ-Ir(ppy)₂in dichloromethane, and the resulting solution is refluxed under nitrogen for 3 days. After the solution is cooled to room temperature, toluene is added, the volume is reduced by rotary evaporation of the methylene chloride, and the solution is cooled for several hours in a freezer. The yellow microcrystalline product is collected by suction filtration, rinsed with 5 mL aliquots of toluene and hexanes, and dried under vacuum to yield the styrenic complex [Ir(ppy)₂(vpy)Cl], as outlined in FIG. 6.

### Example 4

### Vinyl Functional Iridium Singlet Oxygen Generating Monomer

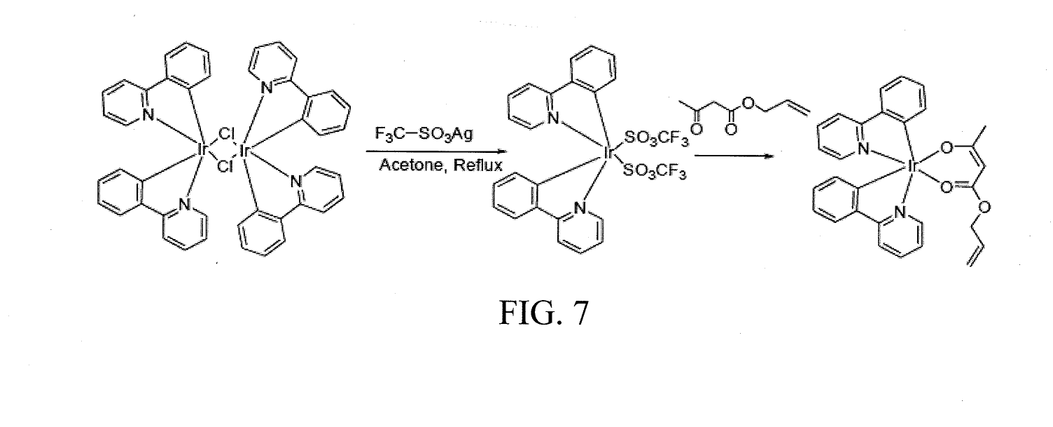

Silver triflate and ((ppy)₂Ir-μ-Cl₂-μ-Ir(ppy)₂) are dissolved in acetone (40 mL) and refluxed under nitrogen for 2 hours. The cloudy yellow solution is cooled and gravity-filtered to remove AgCl. Allylacetoacetate (vacac) and triethylamine are then added to the filtrate. The solution is then stirred overnight at room temperature under nitrogen. After solvent stripping to dryness, the dark yellow solid is purified on a short silica gel column, eluting with dichloromethane. The first bright yellow band is collected and brought to dryness using a rotary evaporator. The product is recrystallized by slow diffusion of hexanes into a concentrated dichloromethane solution, yielding a golden yellow microcrystalline solid [Ir(ppy)₂(vacac)], as outlined in FIG. 7.

### Example 5

### Acrylic Functional Platinum Singlet Oxygen Generating Monomer

The present example outlines a method for the preparation of Pt 3-benzothiazol-2-yl-7-diethylamino-chromen-2-1-chloride-bridged dimer. 3-Benzothiazol-2-yl-7-diethylamino-chromen-2-one in 2-ethoxyethanol (9 mL) is added to a solution of K₂PtCl₄in water. The mixture is heated at 80 degrees Centigrade for 48 hours in an inert gas atmosphere. The solid obtained is filtered, washed with water and methanol and dried under vacuum. The yield of bridged dimer is typically 75%.

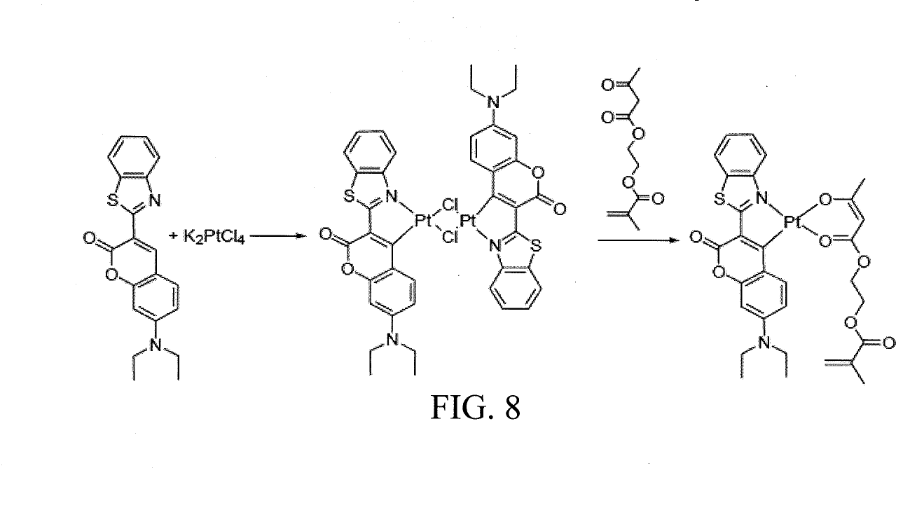

A suspension of thallium 3-oxo-butyric acid 2-(2-methyl-acryloyloxy)-ethyl ester (acrylic acetyl acetate ligand) in dichloromethane is added to a suspension of the dinuclear chloro-bridged Pt(II) complex dissolved in dichloromethane. The resulting mixture is stirred for 180 hours at room temperature. The reaction is monitored by TLC and, after completion, the reaction mixture is filtered through Celite and the solvent removed under by rotary evaporation. Recrystallization of the crude product from chloroform-methanol solution results in a yellow solid, as outlined in FIG. 8.

### Example 6

### Polymerization Of Acrylic Iridium Singlet Oxygen Generating Monomers Into Polymers

A copolymer of Ir(MeBTP)AAc (from Example 4), (trimethylsiloxy)silylpropylmethacrylate (oxygen permeability), and methyl methacrylate (hardness) is synthesized at 75 degrees Celsius, using 2,2′-azobis(isobutyronitrile) (AIBN) as an initiator in tetrahydrofuran (THF) solution. The resultant mixture is dissolved in chloroform, precipitated by pouring into methanol, washed with methanol, and then dried under reduced pressure to yield the polymer material. The reaction scheme is generally outlined in FIG. 9.

### Example 7

### Fibers from Polymer Singlet Oxygen Generating Polymers

Fibers are produced by the use of spinnerets. The fibers can be produced by either dry spinning or wet spinning techniques.

The wet spinning technique is used when fiber-forming polymers have been dissolved in a solvent. The spinnerets are submerged in a chemical bath that contains a solvent that the polymers (from Example 6) are not soluble in. As the polymer filaments emerge from the spinet they precipitate from solution and solidify to form the fiber.

Alternatively, one can employ the dry spinning technique. The polymers (from Example 6) are dissolved in solvents; however, instead of precipitating the polymer by immersion in a non-solvent, solidification is achieved by evaporating the solvent by stream of air or inert gas. Thus, the filaments do not come in contact with a precipitating liquid, eliminating the need for removing the non-solvent and making solvent recovery easier. The dry process can be used for the production of acetate, triacetate, acrylic, and many other commercial polymers.

Alternatively, polymer melts are used to form fibers by forcing a polymer melt (of the polymer of Example 6) through the spinnerets.

### Example 8

### Filters From Singlet Oxygen Generating Fibers

In some embodiments, the fibers, which can be produced as outlined in Example 7, are then woven into a filtration material, and can be used as HEPA type filtration media. The woven singlet oxygen generating materials are then formed into the desired shape of the air filter.

In the alternative, the filter can be produced through the packing of bead materials and sintering them into self-supporting templates. The packing of the nano/microspheres that constitute the template occurs by introducing them into a mold of the shape desired. The interstitial space is then filled with the new reactive materials, which are then polymerized (for example, the compounds of Examples 1-3, or 5). The template is then removed forming the reactive air filter based upon the singlet oxygen generating materials.

### Example 9

### Air Purification Via a Singlet Oxygen Generating Filter

The first filter described from Example 8 is provided and placed in an air intake register. The surface of the filter is exposed to a spectrum of light within a wavelength of 400 to 450 nm resulting in the triplet state, which then interacts with oxygen to produce singlet oxygen. The singlet oxygen then breaks down at least one biological contaminant present in the air that is proximal to or passes through the filter.

### Example 10

### Air Purification Via a Singlet Oxygen Generating Filter

The second filter described from Example 8, involving a polymer of the monomer shown in Example 1, is provided and placed in a water intake register. The surface of the filter is exposed to light having a wavelength of about 425 nm resulting in the triplet state in the singlet oxygen generating moiety, which then interacts oxygen to produce singlet oxygen. Water is then passed through the filter. The singlet oxygen then breaks down a chemical contaminants present in the water that passes through the filter.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. An air filter comprising a polymer fiber comprising a polymer comprising a monomer unit of:
a material capable of excited state energy transfer; and
a polymerized moiety covalently attached to the material capable of excited state energy transfer.

2. The air filter of claim 1, wherein the material capable of excited state energy transfer comprises a material capable of causing a triplet state to be excited in oxygen.

3. The air filter of claim 2, wherein the material capable of causing a triplet state comprises at least one of a metal or an organic molecule.

4. The air filter of claim 3, wherein the metal comprises at least one of iridium, copper, nickel, tin, lead, europium, gadolinium, samarium, terbium, neodymium, thorium, uranium, rhenium, osmium, ruthenium, rhodium, platinum, silver, palladium, gold, cadmium, or mercury.

5. The air filter of claim 3, wherein the metal comprises iridium.

6. The air filter of claim 3, wherein the organic molecule comprises at least one of coumarin, fluorescein, or rhodamine.

7. The air filter of claim 6, further comprising iodine, bromine, selenium, tellurium, or a combination thereof, attached to the organic molecule.

8. The air filter of claim 1, wherein the polymerized moiety comprises at least one polymerized molecule from the group of: an acrylic group, a styrenic group, polyurethane, polyurea, or a vinyl group.

9. (canceled)

10. The air filter of claim 1, wherein the fiber is positioned in a filter.

11. The air filter of claim 10, wherein the filter is one of: a commercial air filter, a residential air filter, a catalytic converter, or a water filter.

12. A method for decontaminating a volume of material, the method comprising:
providing a polymer comprising a monomer unit comprising:
a material capable of excited state energy transfer; and
a polymerized moiety covalently attached to the material capable of excited state energy transfer;
generating at least one singlet oxygen from the polymer and oxygen; and
contacting a volume of material with the singlet oxygen, thereby decontaminating the volume of material.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. The method of claim 12, further comprising enriching an amount of oxygen in the volume of material.

25. (canceled)

26. The method of claim 12, further comprising applying an electrical potential to the volume of material, wherein the material comprises air.

27. The method of claim 26, wherein the electrical potential is applied by a conductive polymer in a filter of which the polymer is part.

28. A method of making a polymer fiber, the method comprising:
providing a polymer comprising a material capable of excited state energy transfer covalently attached to a polymerized moiety; and
forming one or more fiber from the polymer.

29. The method of claim 28, wherein forming the one or more fiber comprises a wet spinning technique.

30. (canceled)

31. (canceled)

32. (canceled)

33. A polymerizable monomer comprising the structure as represented in Formula I, Formula II, Formula III, Formula IV, Formula V, Formula VI, Formula VII, or Formula VIII:

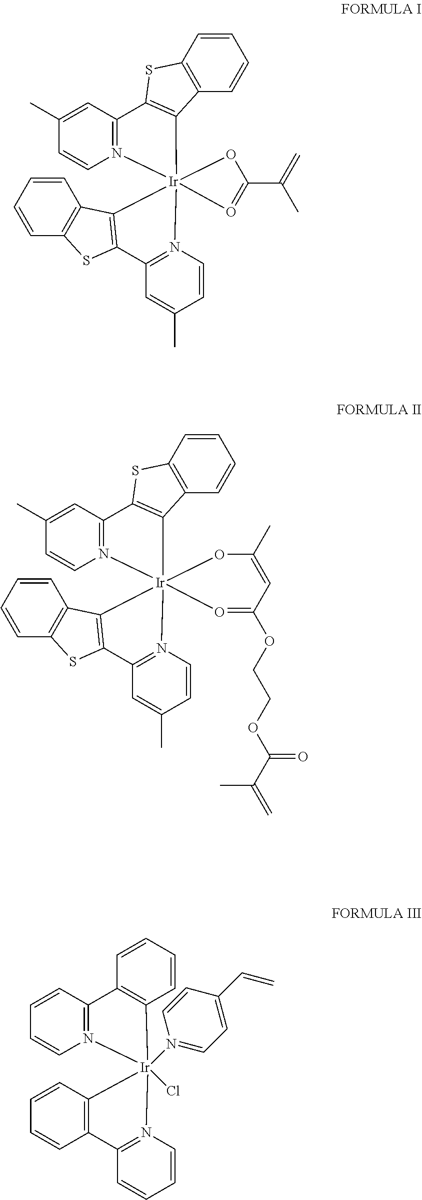

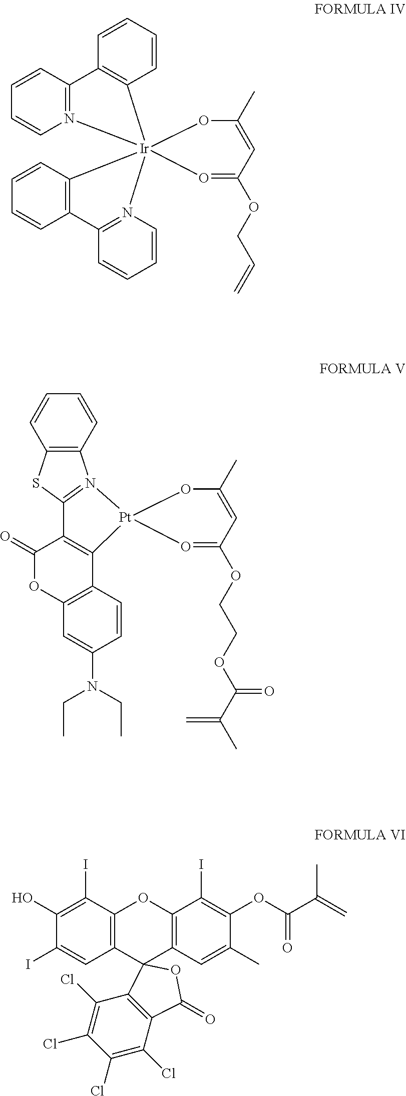

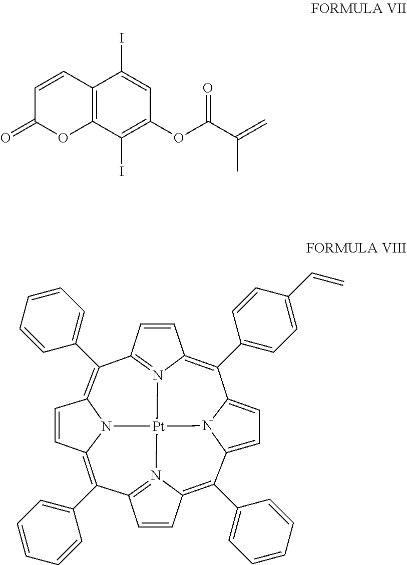

34. A polymer comprising one or more monomer units, wherein the monomer units are represented by Formula IX:

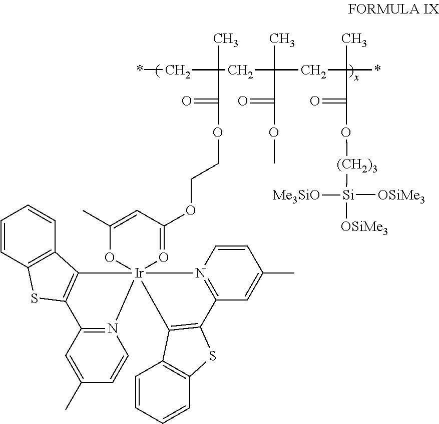

35. A polymer fiber comprising a polymer comprising a monomer unit of:
a material capable of transferring energy from a triplet state of the material to a triplet state of oxygen; and
a polymerized moiety covalently attached to the material.

36. A polymer fiber comprising a polymer comprising a monomer unit of:
a singlet oxygen generating material; and
a polymerized moiety covalently attached to the singlet oxygen generating material.

37. An air filter material comprising of a material comprising a metal chelate moiety of Formula XXXIII 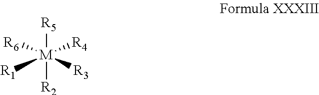
wherein M is at least one of iridium, copper, nickel, tin, lead, europium, gadolinium, samarium, terbium, neodymium, thorium, uranium, rhenium, osmium, ruthenium, rhodium, platinum, silver, palladium, gold, cadmium, or mercury; wherein R₁, R₂, R₃, R₄, R₅, and R₆ are each independently selected from the group consisting of B, C, N, O, F, Si, P, S, Cl, Ge, As, Se, Br, Sn, Sb, Te, and I, wherein one or more of R₁, R₂, R₃, R₄, R₅, and R₆ can be covalently bonded to one or more of R₁, R₂, R₃, R₄, R₅, and R₆; and
wherein at least one of R₁, R₂, R₃, R₄, R₅, and R₆ is attached to a polymerizable moiety or part of an entity attached to a polymerizable moiety.

38. The air filter material of claim 37, wherein the covalent bond of one or more of R₁, R₂, R₃, R₄, R₅, and R₆ to one or more of R₁, R₂, R₃, R₄, R₅, and R₆ at least partially surrounds M in a linear, bent, trigonal planar, square planar, tetrahedral, trigon bipyramidal, octaheadral, pentagonal bipyramidal, square antiprismatic, bisdisphenoid, or hexagonal bipyramidal arrangement.

39. The air filter material of claim 37, wherein the polymerizable moiety comprises at least one molecule from the group of: an acrylic group, a styrenic group, polyurethane, polyurea, or a vinyl group.

40. (canceled)

41. (canceled)

42. (canceled)
